(19) United States
(12) Patent Application Publication
(10) Pub. No.: US 2006/0240108 A1
(43) Pub. Date: Oct. 26, 2006

(54) Cellulosic films incorporating a pharmaceutically acceptable plasticizer with enhanced wettability

(76) Inventor: Bobby Lynn Bernard, Rogersville, TN (US)

Correspondence Address: Michael J. Blake;Eastman Chemical Company, P.O. Box 511, Kingsport, TN, 37662-5075, US

(21) Appl. No.: 11/114,322

(22) Filed: Apr. 26, 2005

(51) Int. Cl. A61K 9/24; A61K 31/717

(52) U.S. Cl. 424/472; 514/57

(57) ABSTRACT

An enteric coating for a solid pharmaceutical carrier or substrate wherein the enteric coating includes a cellulosic polymeric material selected from selected from the group consisting of hydroxypropyl cellulose, hydroxyethyl cellulose, hydroxypropyl methyl cellulose, methyl cellulose, ethyl cellulose, cellulose acetate, cellulose acetate butyrate, cellulose acetate phthalate, cellulose acetate succinate, cellulose acetate propionate, cellulose acetate trimellitate, hydroxypropylmethyl cellulose phthalate, hydroxypropylmethyl cellulose succinate, hydroxypropylmethyl cellulose acetate succinate, cellulose acetate succinate butyrate, cellulose acetate succinate propionate, carboxymethylcellulose sodium, cellulose butyrate, and mixtures thereof and a plasticizer selected from a water-soluble preparation of a fat-soluble vitamin. A preferred plasticizer is Vitamin E polyethylene glycol 1000 succinate.

### BACKGROUND OF THE INVENTION

The present invention relates to drug delivery and more specifically to an enteric coating for pharmaceutical dosage forms for oral administration of a medicament. More particularly, the enteric coating includes a cellulosic ester having incorporated therein a tocopheryl derivative as a plasticizer.

Cellulose esters are well known in the art, as are methods for making cellulose esters, see Kirk-Othmer Encyclopedia of Chemical Technology, 4^{th} edition, vol. 5, pages 496-529, the disclosure of which is incorporated herein by reference. Cellulose esters are widely used in diverse commercial applications. For example, U.S. Pat. No. 6,828,089 discloses the use of cellulose esters for photographic substrates; U.S. Pat. No. 6,828,006 discloses the use of cellulose esters in liquid crystal displays; and U.S. Pat. No. 6,821,602 discloses the use of cellulose esters in magnetic recording media.

In the pharmaceutical area, enteric coating a pharmaceutically active agent is not new. The enteric coating provides for a controlled release of the active agent in a manner that the drug release is accomplished at a predictable location in the lower intestinal tract below the point at which the drug would be released without the coating. The enteric coating also prevents the exposure of the active agent and any excipient or carrier the epithelial and mucosal tissue of the buccal cavity, pharynx, esophagus, and stomach as well as to the enzymes associated with these tissues. The enteric coating therefore helps to protect the active agent and a patient's internal tissue from any adverse event prior to drug release at the desired site of delivery. It has been suggested that multiple enteric coatings may be used to target the release of the active agent at various regions in the lower gastrointestinal tract.

Typically, the enteric coating is a polymeric material. Moreover, the enteric coating usually includes a plasticizer to prevent the formation of pores and cracks that would allow the penetration of the gastric fluids. For example, U.S. Pat. No. 6,468,559 issued to Chen et al. on Oct. 22, 2002 discloses an enterically coated capsule housing a therapeutically effective amount of an active agent selected from bisphosphonic acids and pharmacologically acceptable salts, hydrates and other derivatives thereof in a pharmaceutically acceptable liquid or semi-solid carrier. The enteric coating is selected from cellulosic polymers such as hydroxypropyl cellulose, hydroxyethyl cellulose, hydroxypropyl methyl cellulose, methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, preferably formed from acrylic acid, methacrylic acid, methyl acrylate, ammonio methylacrylate, ethyl acrylate, methyl methacrylate and/or ethyl methacrylate; vinyl polymers and copolymers such as polyvinyl pyrrolidone, polyvinyl acetate, polyvinylacetate phthalate, vinylacetate crotonic acid copolymer, and ethylene-vinyl acetate copolymers; and shellac. The patent discloses that suitable plasticizers include triethyl citrate, glyceryl triacetate, acetyl triethyl citrate, polyethylene glycol 400, diethyl phthalate, tributyl citrate, acetylated monoglycerides, glycerol, fatty acid esters, propylene glycol, and dibutyl phthalate.

Tocopheryl derivatives are well known in the art. For example, U.S. Pat. No. 2,680,749, the entire disclosure of which is incorporated herein by reference, discloses a water-soluble preparation of a fat-soluble vitamin. Generally, the water-soluble tocopherol derivatives are prepared by esterifying a tocopheryl acid ester with polyethylene glycol. A preferred water-soluble preparation of a fat-soluble vitamin is vitamin E succinate polyethylene glycol 1000 available from Eastman Chemical Company under the tradename Vitamin E 1000 TPGS™. Tocopheryl derivatives have been used as: solubilizing emulsifiers, such as disclosed in U.S. Pat. No. 6,416,793 issued to Zeligs et al. on Jul. 9, 2002 or U.S. patent application Ser. No. 20020176894 published Nov. 28, 2002; solubilizing surfactants, such as disclosed in U.S. Pat. No. 6,569,463 issued to Patel et al. on May 27, 2003, the entire disclosures of which are incorporated herein by reference.

A problem with previously known enteric polymeric coating plasticizers is that the plasticizer may interfere with the disintegration or absorption of the active agent when released. Accordingly, there is a need for an enteric coating that includes a plasticizer that would not interfere with the disintegration or absorption of the active agent when released.

### SUMMARY OF THE INVENTION

Briefly, the present invention is an enteric coating for the oral administration of a pharmaceutical dosage or active agent. The enteric coating includes a cellulosic polymeric material and a plasticizer selected from water-soluble preparation of a fat-soluble vitamin.

It is an object of the present invention to provide a cellulosic enteric coating or encapsulating coating that includes a plasticizer that does not interfere with the disintegration or absorption of the active agent when released.

These and other objects and advantages of the present invention will become more apparent to those skilled in the art in view of the following description. It is to be understood that the inventive concept is not to be considered limited to the constructions disclosed herein but instead by the scope of the appended claims.

### DETAILED DESCRIPTION OF THE INVENTION

In accordance with the present invention, a substrate formed from a solid pharmaceutical dosage having an active agent is entrically coated. The substrate utilized in the present invention can be a powder or a multiparticulate, such as a granule, a pellet, a bead, a spherule, a beadlet, a microcapsule, a millisphere, a nanocapsule, a nanosphere, a microsphere, a platelet, a minitablet, a tablet or a capsule. A powder constitutes a finely divided (milled, micronized, nanosized, precipitated) form of an active ingredient or additive, molecular aggregates or a compound aggregate of multiple components or a physical mixture of aggregates of an active ingredient and/or additives. It should be emphasized that the substrate need not be a solid material, although often it will be a solid.

The substrate generally includes a pharmaceutically active agent, a carrier and may also include one or more additives that facilitate the formation of a solid pharmaceutical dosage. The pharmaceutical active agent suitable for use in the present invention is not particularly limited. The active ingredient can be hydrophilic, lipophilic, amphiphilic or hydrophobic, and can be solubilized, dispersed, or partially solubilized and dispersed in a suitable pharmaceutical carrier or excipient. Such active ingredients can be any compound or mixture of compounds having therapeutic or other value when administered to an animal, particularly to a mammal, such as drugs, nutrients, cosmeceuticals, diagnostic agents, nutritional agents, and the like. It should be understood that the categorization of an active ingredient as hydrophilic or hydrophobic may change, depending upon the particular salts, isomers, analogs and derivatives used.

For the purpose of the present invention, hydrophobic active ingredients are compounds with little or no water solubility. Intrinsic water solubility (i.e., water solubility of the un-ionized form) for a hydrophobic active ingredient is less than about 1% by weight, preferably less than about 0.1% and more preferably less than about 0.01% by weight.

In another embodiment, the active ingredient can be hydrophilic. Amphiphilic compounds are also included within the class of hydrophilic active ingredients. Apparent water solubility for a hydrophilic active ingredient is greater than about 0.1% by weight, and preferably greater than about 1% by weight. As one skilled in the art will understand, the hydrophobic active ingredient and hydrophilic active ingredient are not limited by any therapeutic category and include, but are not limited to, analgesics, anti-inflammatory agents, anthelmintics, anti-arrhythmic agents, anti-bacterial agents, anti-viral agents, anti-coagulants, anti-depressants, anti-diabetics, anti-epileptics, anti-fungal agents, anti-gout agents, anti-hypertensive agents, anti-malarials, anti-migraine agents, anti-muscarinic agents, anti-neoplastic agents, erectile dysfunction improvement agents, immunosuppressants, anti-protozoal agents, anti-thyroid agents, anxiolytic agents, sedatives, hypnotics, neuroleptics, β-Blockers, cardiac inotropic agents, corticosteroids, diuretics, anti-parkinsonian agents, gastro-intestinal agents, histamine receptor antagonists, keratolytics, lipid regulating agents, anti-anginal agents, cox-2 inhibitors, leucotriene inhibitors, macrolides, muscle relaxants, nutritional agents, opioid analgesics, protease inhibitors, sex hormones, stimulants, muscle relaxants, anti-osteoporosis agents, anti-obesity agents, cognition enhancers, anti-urinary incontinence agents, nutritional oils, anti-benign prostate hypertrophy agents, essential fatty acids, non-essential fatty acids, and mixtures thereof. In addition to the above, hydrophilic active ingredient can be a cytokine, a peptidomimetic, a peptide, a protein, a toxoid, a serum, an antibody, a vaccine, a nucleoside, a nucleotide, a portion of genetic material, a nucleic acid, and mixtures thereof.

The formulations may further contain additional pharmaceutically acceptable carriers or excipients as appropriate, such as, thickeners, flavoring agents, diluents, emulsifiers, dispersing aids, carrier substances, lubricants or binders. As used herein, the term(s) “pharmaceutical carrier” or “excipient” are used interchangeably to mean a pharmaceutically acceptable solvent, suspending agent or any other pharmacologically inert vehicle in the pharmaceutical formulations for delivering one or more active agents. The excipient may be liquid or solid and is selected with the planned manner of administration in mind, and to provide for the desired bulk, consistency, and delivery effect when combined with the active agent and any other components of a given pharmaceutical composition. Typical pharmaceutical carriers include, but are not limited to, binding agents (e.g., pregelatinised maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, and sodium starch glycolate); wetting agents; diluents; coloring agents; emulsifying agents; pH buffering agents; preservatives; and mixtures thereof.

The substrate may further contain a surfactant. The surfactant may be hydrophilic or lipophilic. The terms “hydrophilic” and “lipophilic” or “hydrophobic” are relative terms. To function as a surfactant, a compound must necessarily include polar or charged hydrophilic moieties as well as non-polar lipophilic (hydrophobic) moieties; that is, a surfactant compound must be amphiphilic. An empirical parameter commonly used to characterize the relative hydrophilicity and hydrophobicity of non-ionic amphiphilic compounds is the hydrophilic-lipophilic balance or “HLB” value. Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having a HLB value greater than about 10, as well as anionic, cationic, or compounds for which the HLB scale is not generally applicable. Similarly, hydrophobic surfactants are compounds having a HLB value less than about 10. It should be appreciated that the HLB value of a surfactant is merely a rough guide generally used to enable formulation of industrial, pharmaceutical and cosmetic emulsions. Additionally, commercial surfactant products are generally not pure compounds, but are complex mixtures of compounds, and the HLB value reported for a particular compound may more accurately be characteristic of the commercial product of which the compound is a major component. Different commercial products having the same primary surfactant component can, and typically do, have different HLB values. In addition, a certain amount of lot-to-lot variability is expected even for a single commercial surfactant product. Keeping these inherent difficulties in mind, and using HLB values as a guide, one skilled in the art can readily identify surfactants having suitable hydrophilicity or hydrophobicity for use in preparing a suitable substrate for the enteric coating of the invention.

The type and amounts of the particular additives present in the substrate will typically depend on processes involved in preparing the solid carrier, the encapsulating coating, or the pharmaceutical dosage form. These processes include agglomeration, air suspension chilling, air suspension drying, balling, coacervation, comminution, compression, pelletization, cryopelletization, extrusion, granulation, homogenization, inclusion complexation, lyophilization, nanoencapsulation. melting, mixing, molding, pan coating, solvent dehydration, sonication, spheronization, spray chilling, spray congealing, spray drying, or other processes known in the art. It is also contemplated that the additive be pre-coated or encapsulated prior to admixing with the active agent.

Compressed tablets may be prepared by compressing, in a suitable machine, the active ingredients in a free-flowing form such as a powder or granules, optionally mixed with other materials, such as a binder (e.g., gums such as tragecanth, acacia, carrageenan), a lubricant (e.g., stearates such as magnesium stearate), a glidant (e.g., talc, colloidal silica dioxide), an inert diluent, a preservative, and/or a surface active or dispersing agent. Preferred binders/disintegrants include EMDEX (dextrate), PRECIROL (triglyceride), PEG, and AVICEL (cellulose). The tablets may optionally be coated or scored and may be formulated so as to provide slow or controlled release of the active ingredients therein

Formulations for oral administration include powders, granules, suspensions, aqueous and non-aqueous solutions, capsules, sachets, troches, tablets, and soft elastic capsules or “caplets”. The substrate compositions may be formulated in a conventional manner using known techniques. The substrate compositions can then be converted using known techniques into the customary unit dosage form formulations, such as tablets, coated tablets, pills, granules, capsules, emulsions, suspensions and solutions. For example, molded tablets may be made by uniformly and intimately bringing into association the active ingredients with liquid carriers or finely divided solid carriers or both, and then, if necessary, shaping the product in a suitable machine.

The therapeutically active agent(s) can be present in a concentration of about 0.5% to about 95% by weight of the total mixture but is generally formulated to provide a therapeutically effective amount of the active agent. The term “therapeutically effective amount”, as used herein, refers to the amount of an active agent which is effective to achieve an intended purpose while avoiding or minimizing undesirable side effects (such as toxicity, irritation or allergic response). Generally, the dosage required to provide an effective amount of an active agent will vary depending on the age, health, physical condition, weight, type and extent of the disease or disorder of the recipient, frequency of treatment, the nature of concurrent therapy (if any) and the nature and scope of the desired effect(s).

In accordance with the present invention, the substrate is coated with an enteric coating. The enteric coating is typically, although not necessarily, a polymeric material. Enteric materials may be incorporated within the dosage form or may be a coating substantially covering the entire surface of tablets, capsules or caplets. Preferred enteric coating materials comprise biodegradable or bioerodible, gradually hydrolyzable polymers. The “coating weight”, or relative amount of coating material per capsule, generally dictates the time interval between ingestion and drug release, i.e., a delayed release. The term “delayed release” as used herein refers to the delivery of the active agent to some generally predictable location in the lower intestinal tract so that release of the active agent can be accomplished at a location more distal than what would have been accomplished if there had been no delayed release alterations. Any coatings should be applied to the substrate of a sufficient thickness so that the entire coating does not dissolve in the gastrointestinal fluids at pH below about 5, but does dissolve at pH about 5 and above. The preferred polymers are cellulosic polymers selected form hydroxypropyl cellulose, hydroxyethyl cellulose, hydroxypropyl methyl cellulose, methyl cellulose, ethyl cellulose, cellulose acetate, cellulose acetate butyrate, cellulose acetate phthalate, cellulose acetate succinate, cellulose acetate propionate, cellulose acetate trimellitate, hydroxypropylmethyl cellulose phthalate, hydroxypropylmethyl cellulose succinate, hydroxypropylmethyl cellulose acetate succinate, cellulose acetate succinate butyrate, cellulose acetate succinate propionate, carboxymethylcellulose sodium, cellulose butyrate, and mixtures thereof. Preferably, the cellulosic polymers are selected form cellulose acetate, cellulose acetate butyrate, cellulose acetate phthalate and mixtures thereof.

The enteric cellulosic coating also includes a plasticizer that imparts flexible resiliency to the material to resist fracturing, for example, during tablet curing or aging. In accordance with the present invention, the plasticizer is a tocopheryl derivative, and preferably is a water-soluble preparation of a fat-soluble vitamin such as those disclosed in U.S. Pat. No. 2,680,749. Generally, the water-soluble tocopherol derivatives useful in the present invention are prepared by esterifying any tocopheryl acid ester with polyethylene glycol. The polyoxyethylene glycol moiety has a molecular weight in the range of about 200 to 20,000, preferably of about 400 to about 10,000, more preferably from about 400 to about 1000 and most preferably the water-soluble preparation of a fat-soluble vitamin is vitamin E polyethylene glycol 1000 succinate available from Eastman Chemical Company under the trade name Vitamin E 1000 TPGS™. The amount of water-soluble tocopherol derivative incorporated into the enteric coating is from about 5 weight % to about 80 weight %, preferably from about 10 to about 60 weight %, more preferably from about 15 to about 50 weight %, and most preferably from about 25 weight % to about 50 weight %, wherein the above weight percentages are based on the total weight of the polymeric cellulosic material and the plasticizer.

A solvent dissolution method may be used to prepare the enteric coating. Generally, a solvent-based coating is when the components of the invention are solubilized and/or dispersed in a solvent; preferably, a common solvent for the water-soluble tocopherol derivative and the cellulosic material is utilized. Solvents with a lower melting point than water and higher evaporation numbers are preferred. The materials may be dissolved separately to form separate solutions that are then combined, or dissolved in the same container, to form a final solution. Dissolution of the components is facilitated by rigorous stirring or heating. Colorants and antisticking agents can be employed as needed. Encapsulation can be conducted using traditional methods such as pan coating, air suspension, and fluidized bed techniques. Several formulation factors, such as air supply, temperature, spray rate, spray system, powder feed, and attrition determine the quality of the end product, and one skilled in the art can readily adjust such parameters as needed.

The present invention is illustrated in greater detail by the specific examples presented below. It is to be understood that these examples are illustrative embodiments and are not intended to be limiting of the invention, but rather are to be construed broadly within the scope and content of the appended claims. All parts and percentages in the examples are on a weight basis unless otherwise stated.

The types of polymers investigated in the studies were cellulose acetate (available from Eastman Chemical under trade name cellulose acetate 398-10NF), cellulose acetate butyrate having a viscosity of 57 poise, 29.5% acetyl and a degree of substitution of 2.0 (available from Eastman Chemical under trade name CAB 171-15PG) and cellulose acetate butyrate having a viscosity of 76 poise, 13.5% acetyl of and a degree of substitution of 1.0 (available from Eastman Chemical under trade name CAB 381-20). The total weight percentage of solids contained in each polymer formulation solution was 15%. The weight percentage ratio of polymer:plasticizer used in the film formulation solutions were 100:0, 90:10, 80:20 and 70:30% levels. Film solutions were prepared, deaerated, cast on glass plates using a Gardner Knife, dried to touch at room temperature and 50% relative humility. In the acetone/water solvent system, the ratio, on a weight basis, of acetone to water was 96:4. At least ten films were cast for each composition.

Testing was performed on the following film properties: mechanical properties of % elongation, tensile strength, thermal stability, wettability, and film permeability.

Tests for tensile strength and percent elongation were performed according to ASTM D882.

Thermo-mechanical properties were measured with a Rheometrics RSA II Solids Analyzer (available from Rheometrics, Inc. Piscataway, N.J.), using the method described in the owner's manual, Publication No. 902-00013A, 1991.

Thermal and oxidative stability were measured using a thermogravimetric analyzer model 2950 and using TA Instruments Thermal Analyst 2200 with Thermal Advantage Version 1.1A and Universal V3.8B analysis software. Generally, the procedure records the weight of a substance in a heated environment at a controlled heating rate over a period of time. The change in weight is automatically recorded as the sample is heated, either at a constant temperature or over a programmed rate from 0.1 to 100° C./minute and in either an air or nitrogen atmosphere at a 50 cc/minute purge rate. Each test required a 6 to 25 milligram sample of material.

Wettability and contact angle were measured using a VCA2500 XE Video Contact Angle System, and computer software VCA Optima XE, both from AST Products, Inc. Billerica Ma. 01821. Generally, the angle is determined using a drop of liquid placed on a solid surface. A photograph of the drop profile is used to calculate the contact angle, i.e., the equilibrium angle formed by the tangent to the point of contact at the solid/liquid interface. The contact angle is determined using a set of 5 data points from the droplet. A film of 5 cm×7 cm or less was used for each sample.

Film permeability for each sample was determined according to ASTM E96.

### EXAMPLES 1-3

These examples illustrate that the % elongation of the polymer films were improved by the use of TPGS as a plasticizer in acetone solvent systems. The cellulose acetate butyrate films were strongly influenced by the use of TPGS as a plasticizer. The elongation was increased by the use of TPGS as a plasticizer particularly in the 20 to 30 wt % ranges. The films also showed improvement when TPGS was used as a co-plasticizer. The cellulose acetate butyrate polymer films tested indicated an increase in % elongation than the cellulose acetate polymer film. The results appear in Tables I-III below.

TABLE I
CA 398-10NF % Elongation

Weight % Plasticizer

| Plasticizer | Solvent System | 0 | 10 | 20 | 30 |
|---|---|---|---|---|---|
| PEG 1000 | Acetone | 8.5 | 8.98 | 10.04 | 23.88 |
| PEG/TPGS | Acetone | 8.5 | 6.04 | 10.14 | 27.26 |
| TPGS | Acetone | 8.5 | 4.88 | 5.26 | 26.52 |
| PEG 1000 | Acetone/water | 6.5 | 2.50 | 2.90 | 20.10 |
| PEG/TPGS | Acetone/water | 6.5 | 2.40 | 2.84 | 3.64 |
| TPGS | Acetone/water | 6.5 | 2.10 | 2.77 | 3.66 |

TABLE II
CAB 171-15PG % Elongation

| | | Weight % Plasticizer | | | |
|---|---|---|---|---|---|
| Plasticizer | Solvent System | 0 | 10 | 20 | 30 |
| PEG 1000 | Acetone | 2.68 | 4.72 | 21.74 | 32.64 |
| PEG/TPGS | Acetone | 2.68 | 4.26 | 12.62 | 38.96 |
| TPGS | Acetone | 2.68 | 1.84 | 10.74 | 19.08 |
| PEG 1000 | Acetone/water | 6.30 | 1.80 | 2.46 | 2.90 |
| PEG/TPGS | Acetone/water | 6.30 | 4.42 | 2.68 | 3.96 |
| TPGS | Acetone/water | 6.30 | 1.43 | 0.60 | 0.92 |

TABLE III
CAB 381-20 % Elongation

| | | Weight % Plasticizer | | | |
|---|---|---|---|---|---|
| Plasticizer | Solvent System | 0 | 10 | 20 | 30 |
| PEG 1000 | Acetone | 2.56 | 2.50 | 1.49 | 1.83 |
| PEG/TPGS | Acetone | 2.56 | 2.24 | 26.54 | 29.50 |
| TPGS | Acetone | 2.56 | 2.52 | 14.50 | 47.42 |
| PEG 1000 | Acetone/water | 2.16 | 1.54 | 1.16 | 2.08 |
| PEG/TPGS | Acetone/water | 2.16 | 1.20 | 2.90 | 4.18 |
| TPGS | Acetone/water | 2.16 | 1.40 | 2.70 | 1.32 |

### EXAMPLES 4-6

In Examples 4-6, all films exhibited a decrease in tensile strength when plasticizers were used. The decrease in tensile strength is directly related to the amount of plasticizer used. The results appear in Tables IV-VI below.

TABLE IV
CA 398-10NF Tensile Strength

| | | Weight % Plasticizer | | | |
|---|---|---|---|---|---|
| Plasticizer | Solvent System | 0 | 10 | 20 | 30 |
| PEG 1000 | Acetone | 81.1 | 52.07 | 36.24 | 26.97 |
| PEG/TPGS | Acetone | 81.1 | 68.98 | 32.25 | 30.36 |
| TPGS | Acetone | 81.1 | 52.88 | 45.21 | 32.89 |
| PEG 1000 | Acetone/water | 58.0 | 38.80 | 23.70 | 9.80 |
| PEG/TPGS | Acetone/water | 58.0 | 49.49 | 27.99 | 18.66 |
| TPGS | Acetone/water | 58.0 | 40.79 | 30.45 | 26.47 |

TABLE V
CAB 171-15PG Tensile Strength

| | | Weight % Plasticizer | | | |
|---|---|---|---|---|---|
| Plasticizer | Solvent System | 0 | 10 | 20 | 30 |
| PEG 1000 | Acetone | 50.27 | 47.62 | 30.02 | 16.84 |
| PEG/TPGS | Acetone | 50.27 | 33.82 | 29.88 | 21.82 |
| TPGS | Acetone | 50.27 | 23.68 | 36.99 | 23.17 |
| PEG 1000 | Acetone/water | 44.57 | 25.44 | 22.96 | 11.94 |
| PEG/TPGS | Acetone/water | 44.57 | 40.86 | 5.80 | 13.35 |
| TPGS | Acetone/water | 44.57 | 23.93 | 10.41 | 11.04 |

TABLE VI
CAB 381-20 Tensile Strength

| | | Weight % Plasticizer | | | |
|---|---|---|---|---|---|
| Plasticizer | Solvent System | 0 | 10 | 20 | 30 |
| PEG 1000 | Acetone | 48.4 | 27.3 | 14.83 | 9.08 |
| PEG/TPGS | Acetone | 48.4 | 26.53 | 21.27 | 13.39 |
| TPGS | Acetone | 48.4 | 26.01 | 22.08 | 19.7 |
| PEG 1000 | Acetone/water | 28.08 | 15.06 | 10.36 | 9.66 |
| PEG/TPGS | Acetone/water | 28.08 | 14.5 | 15.45 | 9.41 |
| TPGS | Acetone/water | 28.08 | 17.54 | 21.91 | 10.07 |

### EXAMPLES 7-9

Examples 7-9 illustrates the stability of the films in nitrogen. Stabilities of the films were examined by thermogravitmetric analysis, which is the temperature at which each film sample lost 10% of its weight. It was observed that in the cellulose acetate polymer (CA 398-10NF) TPGS performed better than PEG 1000 in an acetone/water solvent system at all concentration levels and was substantially equal to the performance of PEG 1000 in the acetone solvent system. The results are presented in Tables VII-IX below.

TABLE VII
CA 398-10NF T10 (Nitrogen) Stability/Flexibility

| | | Weight % Plasticizer | | | |
|---|---|---|---|---|---|
| Plasticizer | Solvent System | 0 | 10 | 20 | 30 |
| PEG 1000 | Acetone | 334.23 | 334.90 | 335.80 | 336.90 |
| PEG/TPGS | Acetone | 334.23 | 339.40 | 340.90 | 338.30 |
| TPGS | Acetone | 334.23 | 333.80 | 338.40 | 342.10 |
| PEG 1000 | Acetone/Water | 336.31 | 298.53 | 268.24 | 245.21 |
| PEG/TPGS | Acetone/Water | 336.31 | 338.60 | 342.40 | 341.80 |
| TPGS | Acetone/Water | 336.31 | 336.60 | 337.80 | 339.50 |

TABLE VIII
CAB 171-15PG T10 (Nitrogen) Stability/Flexibility

| | | Weight % Plasticizer | | | |
|---|---|---|---|---|---|
| Plasticizer | Solvent System | 0 | 10 | 20 | 30 |
| PEG 1000 | Acetone | 348.87 | 353.70 | 354.20 | 351.40 |
| PEG/TPGS | Acetone | 348.87 | 354.10 | 355.40 | 355.40 |
| TPGS | Acetone | 348.87 | 355.20 | 356.10 | 357.40 |
| PEG 1000 | Acetone/Water | 349.29 | 348.04 | 342.44 | 334.27 |
| PEG/TPGS | Acetone/Water | 349.29 | 354.50 | 354.60 | 353.40 |
| TPGS | Acetone/Water | 349.29 | 356.20 | 356.70 | 356.50 |

TABLE IX
CA 381-20 T10 (Nitrogen) Stability/Flexibility

| | | Weight % Plasticizer | | | |
|---|---|---|---|---|---|
| Plasticizer | Solvent System | 0 | 10 | 20 | 30 |
| PEG 1000 | Acetone | 350.75 | 353.70 | 354.20 | 351.40 |
| PEG/TPGS | Acetone | 350.75 | 354.10 | 355.40 | 355.40 |
| TPGS | Acetone | 350.75 | 355.20 | 356.10 | 357.40 |
| PEG 1000 | Acetone/Water | 349.83 | 347.88 | 350.83 | 346.51 |
| PEG/TPGS | Acetone/Water | 349.83 | 354.50 | 354.60 | 353.40 |
| TPGS | Acetone/Water | 349.83 | 356.20 | 356.70 | 356.50 |

### EXAMPLES 10-12

Examples 10-12 illustrates the improvement in wettability as demonstrated by the contact angle of water upon the film coatings. The lower the contact angle, the more wettable the coating. It was observed that 30 weight % TPGS dramatically improved the contact angle of coatings, especially in the cellulose acetate film. The results are presented in Tables X-XII below.

TABLE X
CA 398-10NF Contact Angle

| | | Weight % Plasticizer | | | |
|---|---|---|---|---|---|
| Plasticizer | Solvent System | 0 | 10 | 20 | 30 |
| PEG 1000 | Acetone | 66.70 | 53.25 | 50.84 | 46.78 |
| PEG/TPGS | Acetone | 66.70 | 62.64 | 50.13 | 1.00 |
| TPGS | Acetone | 66.70 | 52.77 | 53.37 | 1.00 |
| PEG 1000 | Acetone/Water | 60.00 | 60.10 | 58.00 | 51.90 |
| PEG/TPGS | Acetone/Water | 60.00 | 41.02 | 46.74 | 105.23 |
| TPGS | Acetone/Water | 60.00 | 46.44 | 49.71 | 104.23 |

TABLE XI
CAB 171-15PG Contact Angle

| | | Weight % Plasticizer | | | |
|---|---|---|---|---|---|
| Plasticizer | Solvent System | 0 | 10 | 20 | 30 |
| PEG 1000 | Acetone | 77.73 | 59.61 | 66.9 | 50.58 |
| PEG/TPGS | Acetone | 77.73 | 71.75 | 61.06 | 12.74 |
| TPGS | Acetone | 77.73 | 77.39 | 69.41 | 19.10 |
| PEG 1000 | Acetone/Water | 81.02 | 74.47 | 77.11 | 59.04 |
| PEG/TPGS | Acetone/Water | 81.02 | 61.60 | 60.12 | 105.72 |
| TPGS | Acetone/Water | 81.02 | 43.50 | 29.63 | 105.82 |

TABLE XII
CAB 381-20 Contact Angle

| | | Weight % Plasticizer | | | |
|---|---|---|---|---|---|
| Plasticizer | Solvent System | 0 | 10 | 20 | 30 |
| PEG 1000 | Acetone | 74.51 | 81.11 | 58.25 | 21.36 |
| PEG/TPGS | Acetone | 74.51 | 78.21 | 77.29 | 23.56 |
| TPGS | Acetone | 74.51 | 77.13 | 80.23 | 67.85 |
| PEG 1000 | Acetone/Water | 74.6 | 71.25 | 68.06 | 67.8 |
| PEG/TPGS | Acetone/Water | 74.6 | 93.48 | 100.63 | 90.33 |
| TPGS | Acetone/Water | 74.6 | 88.9 | 105.67 | 86.45 |

### EXAMPLES 13-15

Examples 13-15 illustrate the affects of Vitamin E 1000 TPGS™ on the rate of water vapor transmission (WVTR) through a film. In film coating applications, such as a tablet, the water vapor transmission rate is a measurement of the rate that water will permeate the tablet coating into the tablet. The WVTR is important in an osmotic pump application. It was observed that the use of TPGS 1000 reduced the WVTR, especially in the cellulose acetate polymer. The results are presented in Tables XII-XV below.

TABLE XIII
CA 398-10NF WVTR

| | | Weight % Plasticizer | | | |
|---|---|---|---|---|---|
| Plasticizer | Solvent System | 0 | 10 | 20 | 30 |
| PEG 1000 | Acetone | 1464 | 1022 | 1132 | 1360 |
| PEG/TPGS | Acetone | 1464 | 1338 | 1310 | 1337 |
| TPGS | Acetone | 1464 | 1460 | 1455 | 1548 |
| PEG 1000 | Acetone/Water | 1505 | 1430 | 1830 | 5007 |
| PEG/TPGS | Acetone/Water | 1505 | 1077 | 1451 | 1648 |
| TPGS | Acetone/Water | 1505 | 1305 | 1392 | 1614 |

TABLE XIV
CAB 171-15PG WVTR

| | | Weight % Plasticizer | | | |
|---|---|---|---|---|---|
| Plasticizer | Solvent System | 0 | 10 | 20 | 30 |
| PEG 1000 | Acetone | 860 | 847 | 1112 | 1650 |
| PEG/TPGS | Acetone | 860 | 797 | 1105 | 1630 |
| TPGS | Acetone | 860 | 1065 | 973 | 1623 |
| PEG 1000 | Acetone/Water | 1091 | 1237 | 1433 | 1846 |
| PEG/TPGS | Acetone/Water | 1091 | 1049 | 1088 | 1472 |
| TPGS | Acetone/Water | 1091 | 1134 | 1077 | 1093 |

TABLE XV
CAB 381-20 WVTR

| | | Weight % Plasticizer | | | |
|---|---|---|---|---|---|
| Plasticizer | Solvent System | 0 | 10 | 20 | 30 |
| PEG 1000 | Acetone | 921 | 996 | 1120 | 1600 |
| PEG/TPGS | Acetone | 921 | 1043 | 1174 | 1360 |
| TPGS | Acetone | 921 | 1139 | 1202 | 1337 |
| PEG 1000 | Acetone/Water | 1451 | 1407 | 1579 | 1830 |
| PEG/TPGS | Acetone/Water | 1451 | 1043 | 1174 | 1515 |

Having described the invention in detail, those skilled in the art will appreciate that modifications may be made to the various aspects of the invention without departing from the scope and spirit of the invention disclosed and described herein. It is, therefore, not intended that the scope of the invention be limited to the specific embodiments illustrated and described but rather it is intended that the scope of the present invention be determined by the appended claims and their equivalents. Moreover, all patents, patent applications, publications, and literature references presented herein are incorporated by reference in their entirety for any disclosure pertinent to the practice of this invention.

What is claimed is:

1. An enteric coating for a solid substrate comprising a cellulosic polymeric material and a plasticizer selected from a water-soluble preparation of a fat-soluble vitamin.

2. The enteric coating of claim I wherein the cellulosic polymeric material is hydroxypropyl cellulose, hydroxyethyl cellulose, hydroxypropyl methyl cellulose, methyl cellulose, ethyl cellulose, cellulose acetate, cellulose acetate butyrate, cellulose acetate phthalate, cellulose acetate succinate, cellulose acetate propionate, cellulose acetate trimellitate, hydroxypropylmethyl cellulose phthalate, hydroxypropylmethyl cellulose succinate, hydroxypropylmethyl cellulose acetate succinate, cellulose acetate succinate butyrate, cellulose acetate succinate propionate, carboxymethylcellulose sodium, cellulose butyrate, or mixtures thereof.

3. The enteric coating of claim 1 wherein the plasticizer is a tocopheryl acid esterified with polyethylene glycol.

4. The enteric coating of claim 3 wherein the polyethylene glycol has a molecular weight of from about 200 to about 20,000.

5. The enteric coating of claim 3 wherein the polyethylene glycol has a molecular weight of from about 400 to about 10,000.

6. The enteric coating of claim 3 wherein the polyethylene glycol has a molecular weight of from about 400 to about 1000.

7. The enteric coating of claim 3 wherein the plasticizer is Vitamin E polyethylene glycol 1000 succinate.

8. The enteric coating of claim 7 wherein the plasticizer is present at from about 5 weight % to about 80 weight %, based on the total weight of the cellulosic polymeric material and the plasticizer.

9. The enteric coating of claim 7 wherein the plasticizer is present at from about 10 to about 60 weight %, based on the total weight of the cellulosic polymeric material and the plasticizer.

10. The enteric coating of claim 7 wherein the plasticizer is present at from about 15 to about 50 weight %, based on the total weight of the cellulosic polymeric material and the plasticizer.

11. The enteric coating of claim 7 wherein the plasticizer is present at from about 25 weight % to about 50 weight %, based on the total weight of the cellulosic polymeric material and the plasticizer.

12. A solid pharmaceutical composition comprising a substrate and an enteric coating on the substrate, wherein the enteric coating comprises a cellulosic polymeric material and a plasticizer selected from a water-soluble preparation of a fat-soluble vitamin.

13. The solid pharmaceutical composition of claim 12 wherein the substrate comprises a pharmaceutically active agent and a carrier, wherein the active agent is selected from the group consisting of analgesics, anti-inflammatory agents, anthelmintics, anti-arrhythmic agents, anti-bacterial agents, anti-viral agents, anti-coagulants, anti-depressants, anti-diabetics, anti-epileptics, anti-fungal agents, anti-gout agents, anti-hypertensive agents, anti-malarials, anti-migraine agents, anti-muscarinic agents, anti-neoplastic agents, erectile dysfunction improvement agents, immunosuppressants, anti-protozoal agents, anti-thyroid agents, anxiolytic agents, sedatives, hypnotics, neuroleptics, β-blockers, cardiac inotropic agents, corticosteroids, diuretics, anti-parkinsonian agents, gastro-intestinal agents, histamine receptor antagonists, keratolytics, lipid regulating agents, anti-anginal agents, cox-2 inhibitors, leucotriene inhibitors, macrolides, muscle relaxants, nutritional agents, opioid analgesics, protease inhibitors, sex hormones, stimulants, muscle relaxants, anti-osteoporosis agents, anti-obesity agents, cognition enhancers, anti-urinary incontinence agents, nutritional oils, anti-benign prostate hypertrophy agents, essential fatty acids, non-essential fatty acids, a cytokine, a peptidomimetic, a peptide, a protein, a toxoid, a serum, an antibody, a vaccine, a nucleoside, a nucleotide, a portion of genetic material, a nucleic acid, and mixtures thereof.

14. The solid pharmaceutical composition of claim 12 wherein the cellulosic polymeric material is hydroxypropyl cellulose, hydroxyethyl cellulose, hydroxypropyl methyl cellulose, methyl cellulose, ethyl cellulose, cellulose acetate, cellulose acetate butyrate, cellulose acetate phthalate, cellulose acetate succinate, cellulose acetate propionate, cellulose acetate trimellitate, hydroxypropylmethyl cellulose phthalate, hydroxypropylmethyl cellulose succinate, hydroxypropylmethyl cellulose acetate succinate, cellulose acetate succinate butyrate, cellulose acetate succinate propionate, carboxymethylcellulose sodium, cellulose butyrate, or mixtures thereof.

15. The solid pharmaceutical composition of claim 12 wherein the plasticizer is a tocopheryl acid esterified with polyethylene glycol, and the polyethylene glycol has a molecular weight of from about 200 to about 20,000.

16. The solid pharmaceutical composition of claim 15 wherein the polyethylene glycol has a molecular weight of from about 400 to about 1000.

17. The solid pharmaceutical composition of claim 15 wherein the plasticizer is Vitamin E polyethylene glycol 1000 succinate.

18. The solid pharmaceutical composition of claim 12 wherein the plasticizer is present at from about 10 to about 60 weight %, based on the total weight of the cellulosic polymeric material and the plasticizer.

19. The solid pharmaceutical composition of claim 18 wherein the plasticizer is present at from 25 weight % to about 50 weight %, based on the total weight of the cellulosic polymeric material and the plasticizer.

20. An enteric coating for a solid substrate comprising:
a. a cellulosic polymeric material selected from the group consisting of hydroxypropyl cellulose, hydroxyethyl cellulose, hydroxypropyl methyl cellulose, methyl cellulose, ethyl cellulose, cellulose acetate, cellulose acetate butyrate, cellulose acetate phthalate, cellulose acetate succinate, cellulose acetate propionate, cellulose acetate trimellitate, hydroxypropylmethyl cellulose phthalate, hydroxypropylmethyl cellulose succinate, hydroxypropylmethyl cellulose acetate succinate, cellulose acetate succinate butyrate, cellulose acetate succinate propionate, carboxymethylcellulose sodium, cellulose butyrate, and mixtures thereof; and
b. a plasticizer selected from a water-soluble preparation of a fat-soluble vitamin.

21. The enteric coating of claim 20 wherein the plasticizer is Vitamin E polyethylene glycol 1000 succinate.

22. The enteric coating of claim 21 wherein the plasticizer is present at from about 5 weight % to about 80 weight %, based on the total weight of the cellulosic polymeric material and the plasticizer.

23. The enteric coating of claim 21 wherein the plasticizer is present at from about 25 weight % to about 50 weight %, based on the total weight of the cellulosic polymeric material and the plasticizer.

24. The enteric coating of claim 21 wherein the cellulosic polymeric material is selected from the group consisting of cellulose acetate, cellulose acetate butyrate, cellulose acetate propionate and mixtures thereof.
